(19) United States
(12) Patent Application Publication
(10) Pub. No.: US 2014/0323451 A1
(43) Pub. Date: Oct. 30, 2014

(54) METHODS FOR ADMINISTERING CORTICOSTEROID FORMULATIONS

(71) Applicant: MAP Pharmaceuticals, Inc., Irvine, CA (US)

(72) Inventors: STEPHEN B. SHREWSBURY, Cupertino, CA (US); PAUL S. USTER, Pleasanton, CA (US); ANDREW P. BOSCO, San Francisco, CA (US); THOMAS A. ARMER, Los Altos, CA (US)

(21) Appl. No.: 14/326,197

(22) Filed: Jul. 8, 2014

Related U.S. Application Data
(63) Continuation of application No. 12/268,394, filed on Nov. 10, 2008.
(60) Provisional application No. 61/069,498, filed on Mar. 14, 2008.
(60) Provisional application No. 61/002,645, filed on Nov. 9, 2007.

(51) Int. Cl. A61K 9/00; A61K 31/58

(52) U.S. Cl. CPC A61K 9/007; A61K 31/58
USPC 514/174

(57) ABSTRACT

Described here are methods for the treatment of respiratory conditions using nebulized corticosteroids. The methods administer a dose of corticosteroid twice a day or more with nebulization times of 5 minutes or less. The faster nebulization times improve patient compliance. The methods also employ a lower corticosteroid dose while achieving therapeutic efficacy similar to commercially available formulations. This results in improved patient safety by reducing the systemic exposure of the corticosteroid.

### CROSS-REFERENCE TO RELATED APPLICATIONS

This application claims priority to U.S. Provisional Patent Application Ser. No. 61/002,645, filed Nov. 9, 2007, and U.S. Provisional Patent Application Ser. No. 61/069,498, filed Mar. 14, 2008, which are hereby incorporated by reference in their entirety.

### FIELD

The methods described here are in the field of respiratory medicine. Specifically, methods that administer corticosteroid formulations by nebulization are described. More specifically, methods for the treatment of asthma that administer lower doses of corticosteroid, and which are associated with improved patient compliance and safety are described.

### BACKGROUND

Asthma is a pulmonary condition characterized by airway inflammation, airway hyperresponsiveness, and reversible airway obstruction. During asthmatic episodes, afflicted individuals often experience labored breathing, wheezing, and coughing. These symptoms may be treated with medications such as corticosteroids, which are administered via pressurized metered-dose inhalers (pMDIs) or dry powder inhalers (DPIs). However, certain patient populations, e.g., pediatric, neurologically impaired, or elderly asthmatics, may lack the breath coordination needed for pMDIs or lack the lung capacity needed to use DPIs. Thus, these asthma patients require the administration of therapy via nebulizers.

Formulations that undergo nebulization are dispersed in air to form an aerosol of very fine liquid droplets suitable for inhalation into the lung. Nebulizers typically use compressed air, piezoelectric or servomechanically generated ultrasonic waves, or a vibrating mesh to create the mist of the droplets, and may also have a baffle to remove larger droplets from the mist by impaction. A variety of nebulizers are available for this purpose, such as soft mist nebulizers, vibrating mesh nebulizers, ultrasonic nebulizers, jet nebulizers, and breath-actuated nebulizers. In use, the nebulized formulation is administered to the individual via a mouthpiece or mask.

Low patient compliance is a generally known problem with nebulized drugs. This is primarily due to the amount of time required for nebulizing the drug, which can last up to 30 minutes or longer, depending on such factors as the volume of liquid formulation to be nebulized, the particular active agent being nebulized, the concentration and surface tension of the active agent in the formulation, and the resulting viscosity of the formulation. Other factors include the condition or symptom being treated, and whether the active agent is present as a solution or suspension. Active agent formulations are generally supplied as nominal 2.0 ml volumes with solution or suspension viscosities ranging from that of water, to 100 times the viscosity of water. These typically require about four to about 20 minutes to nebulize, with the nebulization time increasing as the viscosity increases from that of water. If the formulation is a suspension, an additional 15% to 30% longer time is required to nebulize than solution formulations with comparable viscosities due to the added energy required to from droplets containing suspended particulates. Children and adults who become impatient because of lengthy nebulization times often stop treatment prematurely. Drug delivery is often not linear over time, with the bulk of the drug being delivered near the end of the recommended nebulization time. Thus, early termination of treatment can result in a disproportionally decreased delivery of drug. This can lead to further non-compliance since the inadequate dose will likely fail to provide adequate therapy, and thus discourage further use of the nebulizer treatment regimen.

Another issue with nebulizing drugs relates to the amount of drug actually delivered to the lungs. For example, when nebulizing budesonide using a conventional jet nebulizer, the doses of budesonide are those added to the nebulizing device. However, only approximately 40% to 60% of the drug typically leaves the nebulizer, so only approximately 40% to 60% of the nominal dose is delivered to the patient. This is because the drug is delivered constantly, and when the patient is exhaling, the drug leaving the nebulizer will not be delivered to the patient; instead, it will be lost to the environment. Of the amount delivered to the patient, only a fraction is in droplets having diameters in the respirable range (less than approximately 5 microns) which leaves approximately 10% to 20% of the nominal dose delivered to the lungs. To increase the amount of budesonide delivered to the lungs, either the budesonide dose volume or concentration can be increased. In turn, this may lead to higher maximum plasma concentrations, which are associated with a greater risk of systemic side-effects such as cortisol suppression.

Consequently, new methods for administering nebulized drugs would be useful that maximize compliance and therapeutic efficacy while minimizing safety issues or side effects. Specifically, administration methods having faster nebulization times would be desirable to improve patient compliance. Administration methods that result in improved lung deposition (marker of enhanced therapeutic efficacy) without increasing systemic or oropharyngeal exposure (which leads to side effects) would also be desirable.

### SUMMARY

Described here are methods for the treatment of respiratory conditions using nebulized corticosteroids. The methods may include administration of a dose of corticosteroid at least once a day with nebulization times that are faster than commercially available formulations. This administration regimen improves patient convenience by minimizing delivery times as evidenced by time to maximum plasma concentrations, and thus, may improve patient compliance. Further, because of the unexpected pharmacokinetics exhibited by the administered corticosteroid, as further detailed below, a lower dose of the corticosteroid may be used in comparison to commercially available formulations while achieving similar lung dose and therefore therapeutic efficacy. For example, low doses of 0.25 mg or 0.125 mg or less of budesonide, a corticosteroid, may be administered. This results in improved patient safety by reducing the systemic exposure of the corticosteroid.

In one variation, the methods for treating respiratory conditions include administering a dose of a corticosteroid once a day by nebulization for at least one week (7 days), at least two weeks (14 days), at least three weeks (21 days), at least four weeks (28 days), at least five weeks (35 days), or at least six weeks (42 days) or more. For example, the corticosteroid may be administered for at least two months or at least three months or more if required. The corticosteroid may also be administered more frequently, for example, two, three or four times a day.

The methods also include nebulization times that are faster than commercially available corticosteroid formulations. In one variation, the nebulization time is about 5.0 minutes or less. In another variation, the nebulization time is about 4.0 minutes of less. In yet another variation, the nebulization time is about 3.0 minutes or less. In some instances, the nebulization time may be less than about 2.0 minutes.

In one variation, the methods include administering a dose of about 0.30 mg or less of a corticosteroid at least twice a day, wherein the dose is administered by nebulization of a corticosteroid formulation and results in a pharmacokinetic profile characterized by a Tₘₐₓ that is less than about 5 minutes and an AUC_{0-inf} that is 1.5 times the AUC_{0-inf} of the initial dose, or increases by more than 1.5 times the AUC_{0-inf} of the initial dose, when administered for at least 7 days. Here the pharmacokinetic profile may be further characterized by a Cₘₐₓ AUC_{0-inf} ratio that remains approximately constant over a predefined time period, a Cₘₐₓ that is less than about 850 pg/ml, an AUC_{0-inf} that does not exceed 75,000 pg-min/ml, or a combination thereof. As used herein, the term “Cₘₐₓ” is defined as the maximum plasma concentration following administration, and “Tₘₐₓ” is defined as the time to maximum plasma concentration. By “AUC,” it is typically meant “AUC_{0-inf},” which is the total area under the plasma drug concentration-time curve from time zero and calculated to infinity. Here the Cₘₐₓ, Tₘₐₓ and AUC values are generally measured in units of pg/ml, minutes, and pg-min/ml, respectively.

In some variations, the methods for treating respiratory conditions comprise administering a dose of about 0.30 mg or less of budesonide at least twice a day, wherein administration of the budesonide dose results in a pharmacokinetic profile characterized by a Tₘₐₓ that is less than about 5 minutes, a Cₘₐₓ less than about 850 pg/ml, and an AUC_{0-inf} that increases by more than about 1.5 times the AUC_{0-inf} of the initial dose, but does not exceed 75,000 pg-min/ml when administered repeatedly for at least 7 days.

In another variation, for example when a dose of less than about 0.30 mg of a corticosteroid is administered twice a day for at least six weeks, the AUC (representative of the total amount of drug in the blood after a dose) of the administered corticosteroid at least doubles over the six week period. In one variation, the AUC does not exceed about 60,000 pg-min/ml when the AUC at least doubles. In yet another variation, the AUC does not exceed about 40,000 pg-min/ml when the AUC at least doubles. In further variations, the same corticosteroid dose will result in an AUC that triples over a six week period of therapy. However, these increased AUC values are generally less than or equivalent to a commercially available corticosteroid formulation for nebulization, as further elucidated below, whose profile is known to be safe and free of side effects. The methods may also result in an increase in Cₘₐₓ (maximum plasma concentration following administration) values. Despite the increase in maximum plasma concentrations, the administration methods may provide Cₘₐₓ to AUC ratios that are approximately constant over predefined time periods. This tends to indicate that over repeated treatments, an increase in topical surface area is being treated, while keeping systemic exposure to a minimum.

The methods described here may also reduce one or more systemic side-effects of corticosteroids. In one variation, the methods include administering less than about 0.30 mg dose of a corticosteroid twice a day for at least six weeks in an aerosol, wherein the Cₘₐₓ is less than about 850 pg/ml.

Methods for reducing one or more systemic side-effects of corticosteroids comprising administering a dose of about 0.30 mg or less of a corticosteroid twice a day for at least six weeks in an aerosol, wherein the AUC_{0-inf} is less than about 75,000 pg-min/ml are also described.

The dose of corticosteroid that may be administered ranges from about 0.05 mg to about 1.0 mg. In some variations, the dose of corticosteroid is less than about 0.30 mg. In one instance, the dose is about 0.25 mg or less of corticosteroid. In another instance, the dose is about 0.135 mg or less of corticosteroid. An exemplary corticosteroid is budesonide, including derivatives, analogues, and salts thereof. The corticosteroid may be provided in a formulation that also includes surface active agents, stabilizers, buffers and other excipients.

The methods described here may be used to treat patients with various respiratory conditions. As used herein, the terms “treatment or treating” refer to the amelioration, reduction, or prevention of symptoms indicative of a respiratory condition. For example, the methods may be used to treat inflammatory airway conditions such as asthma, chronic obstructive pulmonary disease (COPD), Respiratory Distress Syndrome, chronic cough, and bronchiolitis. Infectious and neoplastic airway conditions are also contemplated. The patients that may be treated can be of any age, ranging from neonates, infants, children, and adolescents (pediatric age groups). The methods may also be useful in adults.

### BRIEF DESCRIPTION OF THE DRAWINGS

FIG. 1 is a graphical representation of the mean plasma budesonide concentration-time profile obtained with nebulization of the 0.25 mg/1.5 ml budesonide formulation shown in Table 6.

FIG. 2 is a graphical representation of the mean maximum plasma concentration obtained with a dose of 0.25 mg budesonide provided by the formulation shown in Table 6 after the first administered dose and after six weeks of treatment (day 42).

FIG. 3 is a graphical representation of the mean plasma budesonide concentration-time profile obtained with nebulization of the 0.135 mg/1.5 ml budesonide formulation shown in Table 6.

FIG. 4 is a graphical representation of the mean maximum plasma concentration obtained with a dose of 0.135 mg budesonide provided by the formulation shown in Table 6 after the first administered dose and after six weeks of treatment (day 42).

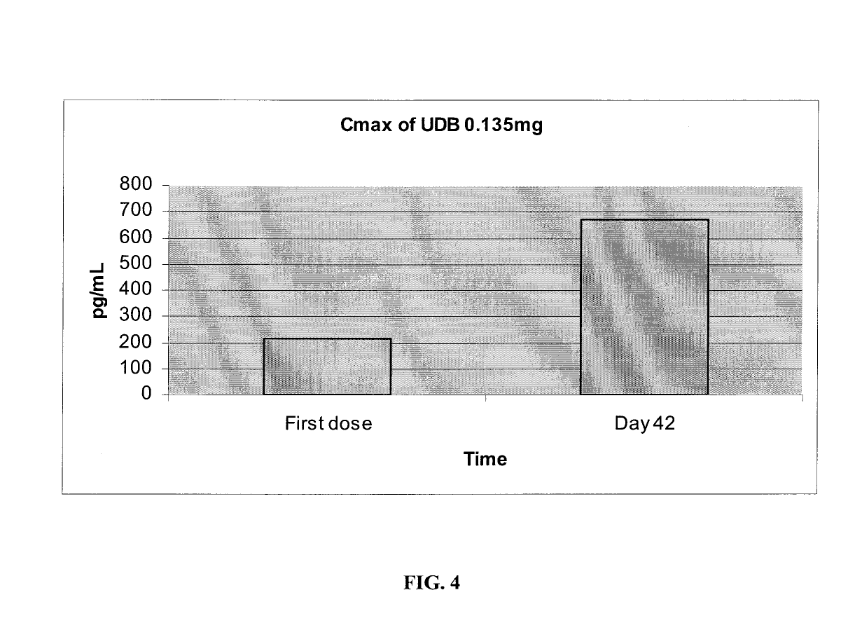

### DETAILED DESCRIPTION

Described here are methods for the treatment of respiratory conditions using nebulized corticosteroids. The methods administer a dose of corticosteroid at least once a day with nebulization times that are faster than commercially available formulations. For example, the nebulization times may be about 5.0 minutes of less, about 4.0 minutes or less, about 3.0 minutes or less, or about 2.0 minutes or less. This administration regimen improves patient convenience, and thus, may improve patient compliance. Further, because of the unexpected pharmacokinetics exhibited by the administered corticosteroid, as further described below, a lower dose of the corticosteroid may be used in comparison to commercially available formulations while achieving similar therapeutic efficacy. This results in improved patient safety by reducing the systemic exposure of the corticosteroid.

The methods described herein administer a corticosteroid at least once a day. However, corticosteroid administration may be repeated or administered more frequently. For example, the corticosteroid may be administered at least two times, at least three times, or at least four times a day. Scheduling may also be varied. For example, the corticosteroid may be administered twice a day for at least one week (7 days), twice a day for at least about two weeks (14 days), twice a day for at least three weeks (21 days), twice a day for at least four weeks (28 days), twice a day for at least 5 weeks (35 days), or twice a day for at least six weeks (42 days) or more. In some variations, the corticosteroid is delivered twice a day every two days, twice a day every three days, twice a day every four days, twice a day every week, twice a day every two weeks, or twice a day every four weeks or more. These regimens may be continued as long as required.

The dose of corticosteroid may also vary, but will generally be a low dose. For example, when budesonide is used, the dose of budesonide that is administered may be less than about 0.30 mg. In one variation, the budesonide dose is between about 0.22 mg to about 0.27 mg. In another variation, the budesonide dose is between about 0.10 mg to about 0.27 mg. In other variations, the dose of budesonide administered is about 0.25 mg or less. In further variations, the dose of budesonide administered is about 0.135 mg or less. In some variations, a nebulizer may be used to generate the aerosol of corticosteroid for administration. Examples of commercially available nebulizers include the AERONEB™ and AERONEB GO™ nebulizers (Aerogen, San Francisco, Calif.); PARI nebulizers, including the PARI LC PLUS™, PART BOY™ N, PARI eflow, PARI LC SINUS, PARI SINUSTAR™, PART SINUNEB, and PARI DURANEB™ nebulizers (PART Respiratory Equipment, Inc., Monterey, Calif.); MICROAIR™ nebulizer (Omron Healthcare, Inc, Vernon Hills, Ill.); HALOLITE™ nebulizer (Profile Therapeutics Inc., Boston, Mass.); RESPIMAT™ nebulizer (Boehringer Ingelheim Ingelheim, Germany); ERODOSE™ nebulizer (Aerogen, Inc., Mountain View, Calif.); OMRON ELITE™ (Omron Healthcare, Inc., Vernon Hills, Ill.); OMRON MICROAIR™ (Omron Healthcare, Inc, Vernon Hills, Ill.); MABISMIST™ II nebulizer (Mabis Healthcare, Inc, Lake Forest, Ill.); LUMISCOPE™ 6610 nebulizer; (The Lumiscope Company, Inc., East Brunswick, N.J.); AIRSEP MYSTIQUE™ nebulizer, (AirSep Corporation, Buffalo, N.Y.); ACORN-1 and ACORN-11 (Vital Signs, Inc, Totowa, N.J.); AQUATOWER™ nebulizer (Medical Industries America, Adel, Iowa); AVA-NEB (Hudson Respiratory Care Incorporated, Temecula, Calif.); AEROCURRENT™ nebulizer utilizing the AEROCELL™ disposable cartridge (AerovectRx Corporation, Atlanta, Ga.); CIRRUS (Intersurgical Incorporated, Liverpool, N.Y.); DART (Professional Medical Products, Greenwood, S.C.); DEVILBISS™ PULMO AIDE (DeVilbiss Corp; Somerset, Pa.); DOWNDRAFT™ (Marquest, Englewood, Colo.); FAN JET (Marquest, Englewood, Colo.); MB-5 (Mefar, Bovezzo, Italy); MISTY NEB™ (Baxter, Valencia, Calif.); SALTER 8900 (Salter Labs, Arvin, Calif.); SIDESTREAM™ (Medic-Aid, Sussex, UK); UPDRAFT-II™ (Hudson Respiratory Care; Temecula, Calif.); WHISPER JET™ (Marquest Medical Products, Englewood, Colo.); AIOLOS™ (Aiolos Medicnnsk Teknik, Karlstad, Sweden); INSPIRON™ (Intertech Resources, Inc., Bannockburn, Ill.); OPTIMIST™ (Unomedical Inc., McAllen, Tex.); PRODOMO™ and SPIRA™ (Respiratory Care Center, Hameenlinna, Finland); AERx™, Essence™, and Ultra™ nebulizers (Aradigm Corporation, Hayward, Calif.); SONIK™ LDI Nebulizer (Evit Labs, Sacramento, Calif.); and SWIRLER® Radioaerosol System (AMICI, Inc., Spring City, Pa.). Exemplary vibrating membrane, mesh or plate nebulizers are described by R. Dhand (Respiratory Care, (December 2002), 47(12), p. 1406-1418).

The respiratory conditions that may be treated with the methods described here include without limitation, asthma, chronic obstructive pulmonary disease (COPD), emphysema, bronchitis, bronchiolitis, pneumonia, neoplasms of the large and small airways, and respiratory distress syndrome. The patients that may be treated can be of any age, ranging from neonates, infants, children, and adolescents (pediatric age groups). The methods may also be useful in adults.

The methods described here can provide more desirable pharmacokinetic parameters than commercially available corticosteroid formulations. An exemplary commercial formulation is Pulmicort Respules® ampules, referred to herein as “Pulmicort Respules.” Pulmicort Respules are manufactured and sold as a budesonide inhalation suspension by Astrazeneca (Wilmington, Del.), and supplied in doses of 0.25 mg, 0.50 mg, and 1.0 mg per 2.0 ml ampules. Presently, Pulmicort Respules are the only FDA approved nebulized corticosteroid product on the U.S. market for the treatment of pediatric asthma. Regardless of the dose, Pulmicort Respules are administered once daily.

Exemplary Method of Treating Respiratory Conditions

In one variation of the method, desirable pharmacokinetic parameters were demonstrated in a randomized, double blind, placebo-controlled, six week study, in which efficacy, safety, and pharmacokinetics of the unit dose budesonide formulations (UDB) shown in Table 6 were assessed in 205 patients aged 4 to 18 years old with asthma. The budesonide was administered twice a day by a nebulizer for six weeks (42 days). In one subset, budesonide was administered at a dose of 0.25 mg. In the other subset, budesonide was administered at a dose of 0.135 mg. Treatment at either dose resulted in improvement of asthma symptoms. Additionally, faster nebulization times were observed as shown in Table 1, with average times of about 4.7 minutes at the beginning of treatment and decreasing to about 3.8 minutes at week six of treatment. Nebulization times for comparable commercial formulations such as Pulmicort Respules are about 8 minutes or more in comparable subjects, as shown in Table 2.

TABLE1
Mean Nebulization Time in Children Ages 2-18 Using
PARI LC Plus Nebulizer

| Mean time to | | | |
|---|---|---|---|
| sputter (mins) | Placebo | UDB 0.135 mg | UDB 0.25 mg |
| Visit 2 | 4.2 | 4.7 | 4.7 |
| Visit 3 | 3.8 | 4.1 | 4.0 |
| Visit 4 | 3.5 | 3.7 | 4.2 |
| Visit 5 | 3.7 | 3.6 | 3.9 |

TABLE 2
Pulmicort Respules Nebulization Time in Various Commercial Nebulizers
For Administration to Children*

| | | Inhalation | Dose | Diameter of |
|---|---|---|---|---|
| | | Time | Delivered | Droplets |
| Nebulizer System | Type | (min) | (% of Nominal) | (μm) |
| Omron NE -U22 | Mesh | 15 | 29 +/− 1.4 | 5.6 +/− 0.24 |
| Omron NE -C16 | Jet | 9-12 | 10 +/− 0.7 | 6.9 +/− 0.16 |
| Pari LC Plus | Jet | 9 | 20 +/− 1.0 | 6.2 +/− .031 |
| Pari LC Plus Junior | Jet | 9 | 18 +/− 0.2 | 7.6 +/− 0.26 |
| Mefat 2000 ( | Jet | 12 | 9 +/− 1.5 | 7.8 +/− 0.70 |
| Azwell Nescojet AZ-11 | Jet | 9 | 10 +/− 1.7 | 7.7 +/− 0.49 |
| Nissho inhaling | Jet | 12 | 15 +/− 2.2 | 9.9 +/− 2.33 |

Compressor

| | | | | |
|---|---|---|---|---|
| DeViibiss PulmoAide | Jet | 9 | 11 +/− 2.1 | 6.5 +/− 0.41 |

5650

| | | | | |
|---|---|---|---|---|
| Milllicon -S | Jet | 12 | 15 +/− 1.4 | 7.4 +/− 0.04 |
| SideStream | Jet | 8 | 4 +/− 0.6 | 5.3 +/− 0.11 |

*data translated for Japanese Ministry of Health Approval of Pulmicort Respules

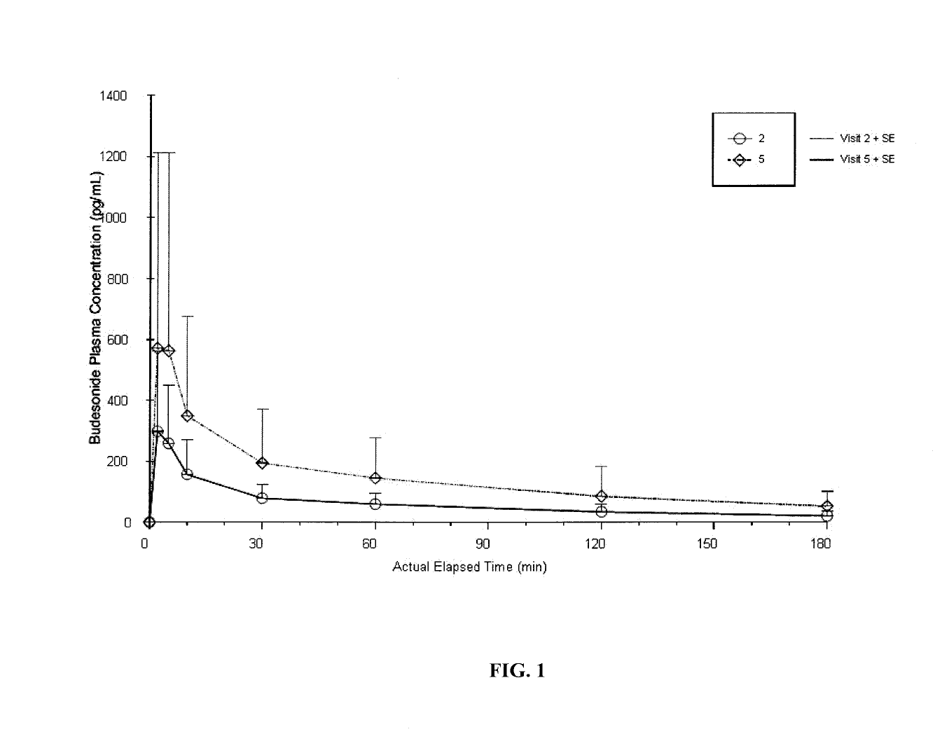

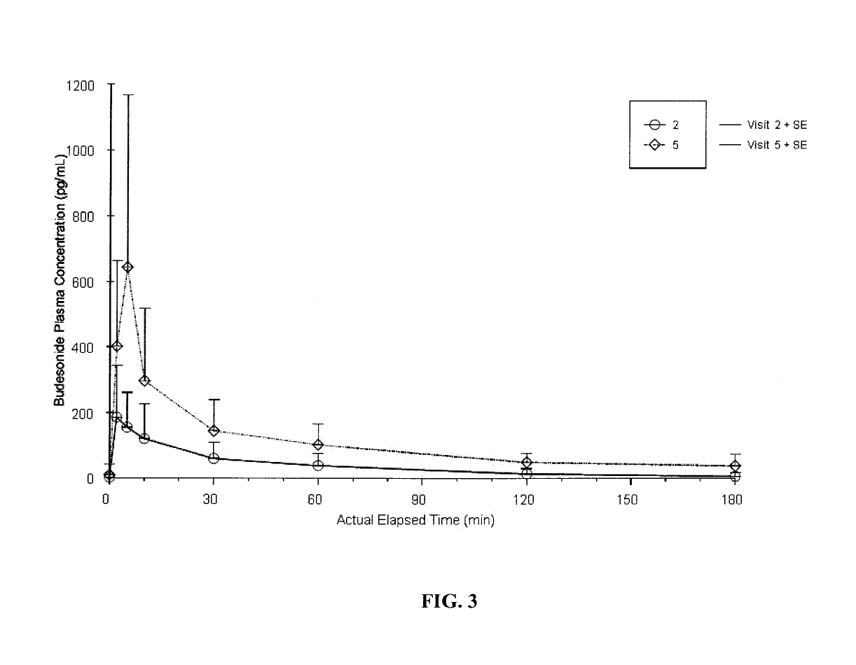

The faster nebulization times shown in Table 1 resulted in shorter Tₘₐₓ (time to maximum concentration of drug in the blood after a dose). Tₘₐₓₛ averaged about 4.3 minutes (SD±0.63 minutes) for the two dosage strengths and time of treatment that ranged from zero to six weeks. As shown in Tables 3 and 4 and FIGS. 1 and 3, maximum plasma concentrations (Cₘₐₓₛ) were also demonstrated in less than about 5 minutes, indicating that Cₘₐₓₛ were being approximately obtained by the end of nebulization. The Cₘₐₓₛ and AUCs (area under the curve) obtained for the two different dosage strengths were below levels that would induce side effects due to systemic exposure. Referring to Tables 3 and 4, Cₘₐₓ ranged from about 670 pg/ml to about 750 pg/ml, and AUC_{0-inf} ranged from about 22,000 pg-min/ml to about 33,000 pg-min/ml. These levels are significantly below those found in a clinical study by Murphy et al. (Murphy, K., Noonan, M., Silkoff, P. Uryniak, T., “A 12 Week Multicenter, Randomized, Partially Blinded, Active Controlled, Parallel-Group Study of Budesonide Inhalation Suspension in Adolescents and Adults with Moderate to Severe Persistent Asthma Previously Receiving Inhaled Corticosteroids with a Metered-Dose or Dry Powder Inhaler”, Clinical Therapeutics, Vol. 29, No, 6, pp. 1013-1026, June 2007). Murphy et al.'s dosing regimen of 1.0 mg twice a day (bid) was the same as that for Pulmicort Respules when the pharmacokinetics of Pulmicort Respules was being assessed by Astrazeneca (Pharmacokinetics section of Pulmicort Respules label insert). At the 1.0 mg bid dose, significant safety issues (suppression of cortisol) were noted by the FDA. Given that UDB administration resulted in lower C, and AUC values than Murphy et al. (and thus, Pulmicort Respules), while having comparable efficacy, it is believed that side-effects would be reduced due to the lower systemic exposure.

TABLE 3
Mean pK Parameters For 0.25 mg Dose Budesonide Administered
To Asthmatic Children Over Six Weeks

| Geometric | 0.25 mg | |
|---|---|---|
| mean PK parameters (units) | First Dose | Day 42 |
| AUC_{0-inf} (pg-min/mL) | 20,849 | 33,116 |
| Tₘₐₓ (min) | 3.3 | 5.0 |
| Cₘₐₓ (pg/mL) | 320.7 | 745.9 |
| T_{1/2} (min) | 154.6 | 75.7 |

TABLE 4
Mean pK Parameters For 0.135 mg Dose Budesonide Administered
To Asthmatic Children Over Six Weeks

| Geometric | 0.135 mg | |
|---|---|---|
| mean PK parameters (units) | First Dose | Day 42 |
| AUC_{0-inf} (pg-min/mL) | 10,238 | 22,071 |
| Tₘₐₓ (min) | 4.4 | 4.4 |
| Cₘₐₓ (pg/mL) | 214.4 | 672.1 |
| T_{1/2} (min) | 42.1 | 71.3 |

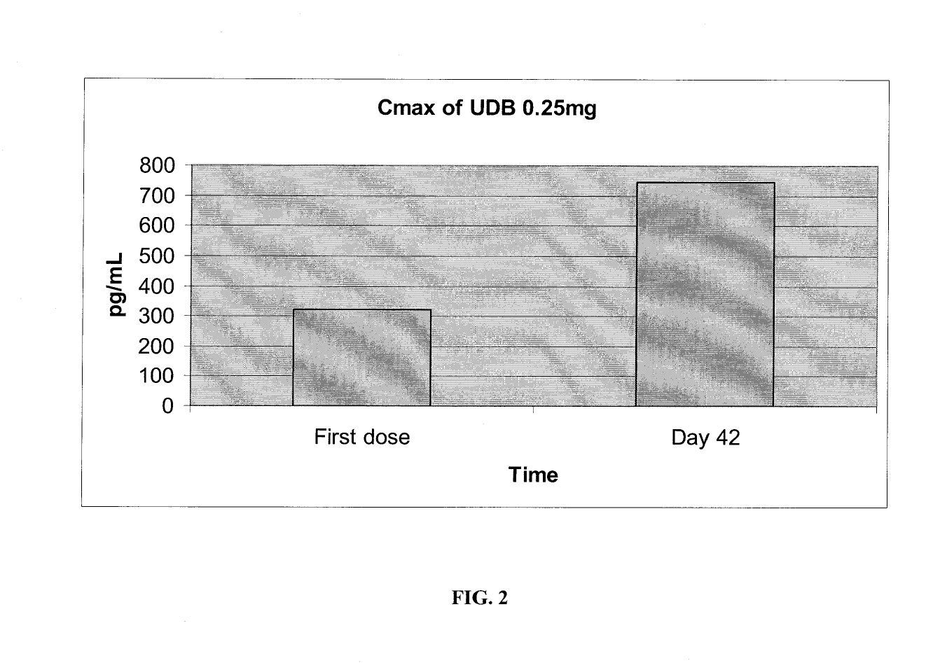

Referring to Table 3 and FIG. 2, AUC_{0-inf} (pg-min/mL) was shown to increase from about 20,849 pg-min/ml at initial dosing to about 33,116 pg-min/ml, about 1.6 times its value at first dose after repeat dosing of 0.25 mg budesonide for six weeks. Cₘₐₓ was shown to at least double after 42 days (six weeks) of dosing.

Referring to Table 4, the Cₘₐₓ for the 0.135 mg dose increased from about 214 pg/ml to about 672 pg/ml from initial dosing to week six of dosing, more than tripling. The corresponding AUC_{0-inf} more than doubled by week six of dosing. Despite the increase in Cₘₐₓ and AUC_{0-inf}, the Tₘₐₓ and the ratios of Cₘₐₓ to AUC_{0-inf} remained relatively constant. The Tₘₐₓ averaged 4.4 minutes over the 6 weeks for the 0.135 mg dose. Within the normal variance between subjects with different ages and weights, the Cₘₐₓ to AUC_{0-inf} ratio remained constant at approximately 0.02 min⁻¹ with a standard deviation less than approximately 0.006 min⁻¹. A similar observation may be made for the 0.25 mg dose where the Tₘₐₓ averaged 4.1 minutes over 6 weeks, and the Cₘₐₓ to AUC_{0-inf} ratio remained relatively constant at approximately 0.025 min⁻¹ with a standard deviation less than approximately 0.007 min⁻¹. Without being bound by theory, it is believed that this indicates that the budesonide is being deposited and absorbed from increasing topical area in the lung over time.

As a comparative example, Table 5 illustrates the pharmacokinetic response to the administration of budesonide in adults. Here the 0.135 mg dose of budesonide (UDB; provided in the formulation shown in Table 6) corresponded to the 0.25 mg dose for Pulmicort Respules, and the 0.25 mg dose of budesonide (UDB; provided in the formulation shown in Table 6) corresponded to the 0.5 mg dose of Pulmicort Respules in intended therapeutic effect. The AUC_{0-inf} of the Pulmicort Respules increased about 1.6 times after 7 days (one week) of repeat dosing twice daily, while that of UDB more than tripled. Despite the tripling of the AUC_{0-inf} for UDB, indicating higher absorption of the budesonide, in no case did the UDB AUC_{0-inf} s exceed those of corresponding doses of the Pulmicort Respules. This indicates that for comparable lung dosing, there is lower systemic exposure because the UDB doses were at approximately half those of the Pulmicort Respules. Given the large increase of AUC_{0-inf}, it is believed that a similar result would have been obtained in children if measured at one week.

TABLE 5
Mean pK Parameters For Pulmicort Respules and UDB Administered
to Asthmatic Adults Over 1 Week

| | Pulmicort Respules | | | |
|---|---|---|---|---|
| | Dose 1 (Day 1) | | Dose 15 (Day 7) | |
| | 0.25 mg | 0.5 mg | 0.25 mg | 0.5 mg |
| AUC_{0-inf} (pg-min/ml) | 26,900 | 55,900 | 45,200 | 90,100 |
| | UDB | | | |
| | Dose 1 (Day 1) | | Dose 15 (Day 7) | |
| | 0.135 mg | 0.25 mg | 0.135 mg | 0.25 mg |
| AUC_{0-inf} (pg-min/ml) | 10,300 | 19,400 | 35,050 | 58,700 |

An important measure of systemic exposure to corticosteroids is the level of endogenous cortisols in the blood. Excess exogenous corticosteroids will suppress natural production of cortisols due to the suppression of the adrenal cortex. Measurement of adrenocorticotropic hormone (ACTH)-induced plasma cortisol levels in children who were administered budesonide as shown in Tables 3 and 4, showed no evidence of hypothalamus-pituitary-adrenal (HPA) axis suppression by budesonide after six weeks of treatment. Specifically, the data demonstrated a change in median plasma cortisol values over six weeks from 11.0 μg/dl to 11.3 μg/dl for the 0.25 mg budesonide dose, and 10.8 μg/dl to 12.0 μg/dl for the 0.135 mg budesonide dose. Both these changes are statistically insignificant. In sum, these results demonstrated the efficacy and safety (reduced systemic exposure/side-effects) of the budesonide formulations provided in Table 6 when administered twice a day.

The data from the clinical trial demonstrated that a dose of about 0.30 mg or less of budesonide at least twice a day may result in a pharmacokinetic profile characterized by a Tₘₐₓ that is less than about 5 minutes and an AUC_{0-inf} that increases by more than about 1.5 times the AUC_{0-inf} of the initial dose when administered for at least 7 days. Here the pharmacokinetic profile may also be further characterized by a Cₘₐₓ to AUC_{0-inf} ratio that remains approximately constant over a predefined time period, a Cₘₐₓ of about 850 pg/ml or less, and an AUC_{0-inf} that does not exceed about 75,000 pg-min/ml.

The data also showed that administering a dose of 0.30 mg or less of budesonide by nebulization twice a day for at least a six week period may result in an AUC_{0-inf} that at least doubles over the six week period. Such administration may also result in a Cₘₐₓ that is less than about 850 pg/ml.

Formulations

Any corticosteroid formulation suitable for nebulization may be used with the methods described here. Suitable corticosteroids that may be employed include, but are not limited to, 21-acetoxypregnenolone, alclometasone, algestone, amcinonide, beclomethasone, betamethasone, budesonide, chloroprednisone, ciclesonide, clobetasol, clobetasone, clocortolone, cloprednol, corticosterone, cortisone, cortivazol, deflazacort, desciclesonide, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolonc, tixocortol, triamcinolone, triamcinolone acetonide, triamcinolone benetonide, triamcinolone hexacetonide, and derivatives, analogues, enantiomer forms, stereoisomers, anhydrides, acid addition salts, base salts, solvates, and combinations thereof. In one variation, the corticosteroid is budesonide.

The formulations may also include excipients and/or additives. Suitable excipients and/or additives that may be employed include one or more surface active agents, phospholipids, solubility enhancers, surface modifiers, antioxidants, chelating agents, or combinations thereof. Useful surface stabilizers include, but are not limited to, non-ionic surface stabilizers such as polyoxyethylene sorbitan esters and polysorbate 80. Useful phospholipids include without limitation, lecithin NF grades or synthetic phospholipids including lecithin NP, purified lecithin, hydrogenated lecithin, soy or egg lecithin phosphatides containing mixtures of anionic phosphatides such as phosphatidylinositol, phosphatidylserine, phosphatidic acid, phosphatidylglycerol, the corresponding lysophosphatides, synthetic phosphatidic acid, and mixtures thereof. Chelating agents include, but are not limited to, cyclodextrins, cromoglycates, xanthates including caffeine, pegylation agents, crown ethers, ethylenediaminetetraacetic acid (EDTA) or a salt thereof, such as the disodium salt, citric acid, nitrilotriacetic acid and the salts thereof. Antioxidants include, but are not limited to, vitamins, provitamins, ascorbic acid, vitamin E, or salts or esters thereof.

Other excipients that may be used, include, but are not limited to, one or more inclusion complexes, pH buffers, tonicity modifiers, binding agents, filling agents, lubricating agents, suspending agents, sweeteners, flavoring agents, preservatives, wetting agents, disintegrants, and effervescent agents.

Examples of suitable preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Exemplary Formulations

Exemplary formulations that may be used with the methods described here may include budesonide and one or more of a surface active agent, a phospholipid, and EDTA. In some variations, the formulations include micronized budesonide, polysorbate 80, hydrogenated soy lecithin, and EDTA. For example, the formulations may comprise between about 0.0031% to about 0.025% by weight micronized budesonide, between about 0.0001% to about 1.0% by weight polysorbate 80, between about 0.00016% to about 0.00125% hydrogenated soy lecithin, and between about 0.0001% to about 5.0% by weight EDTA. In one variation, the formulation includes about 0.005% by weight EDTA. Exemplary budesonide formulations are shown in Table 6. These formulations may be made by the process described in Example 1.

TABLE 6
Exemplary Budesonide (UDB) Formulations

| | Content Per Unit | Content Per Unit |
|---|---|---|
| | Dose (0.135 mg | Dose (0.25 mg |
| Component | budesonide) | budesonide) |
| Budesonide, Micronized | 0.135 mg | 0.250 mg |
| Polysorbate 80 | 0.037 mg | 0.043 mg |
| Hydrogenated Soy | 0.007 mg | 0.013 mg |
| Lecithin | | |
| (S75-3) | | |
| EDTA | 0.075 mg | 0.075 mg |
| (Edetate Disodiurn | | |
| Dihydrate) | | |
| Sodium Chloride | 12.75 mg | 12.75 mg |
| Sodium Citrate | 0.94 mg | 0.94 mg |
| Dihydrate | | |
| Citric Acid | 0.28 mg | 0.28 mg |
| Water for Injection | q.s. to 1.5 ml | q.s. to 1.5 ml |

The invention will be further understood by the following non-limiting examples.

### EXAMPLES

### Example 1

### Preparation of Budesonide Formulations

To form a unit dose budesonide formulation, budesonide particles are initially processed to produce a sterile bulk drug intermediate dispersion, which is further processed into a final aerosol formulation. In the initial processing, the crystalline budesonide starting material is subjected to a milling step to reduce the size of the budesonide particles. The milling step is accomplished by milling crystalline budesonide starting material in a dilute solution of polysorbate 80 (Tween 80) and a milling media to a substantially smaller diameter. The budesonide particles produced are stabilized by the subsequent addition of hydrogenated soy lecithin and disodium edetate. The resulting concentrated bulk drug intermediate dispersion is then sterilized. The bulk drug intermediate dispersion is further processed into a desired aerosol formulation by diluting it aseptically to the appropriate strength by addition of a sterile citrate-buffered isotonic saline solution. The final pH of the aerosol formulation may be from about pH 4 to about pH 7. It is understood that the amount of sodium citrate or citric acid added for dilution may be modified to produce the desired pH.

1-46. (canceled)

47. A method for treating a respiratory condition, the method comprising administering by nebulization a dose of medication, wherein a dose of the medication comprises polysorbate 80, hydrogenated soy lecithin, EDTA, sodium chloride, sodium citrate dihydrate, citric acid, and 0.25 mg or less of micronized budesonide, and results in an AUC_{0-inf} that increases by more than 1.5 times the AUC_{0-inf} of the initial dose when the dose of the medication is administered at least twice a day for at least 7 days, and wherein the nebulization time is 5.0 minutes or less.

48. The method of claim 47, wherein a T max is the same time as the nebulization time of the medication.

49. The method of claim 47, wherein the dose of the medication comprises 0.135 mg or less of micronized budesonide.

50. The method of claim 47, wherein the pulmonary condition is asthma.

51. The method of claim 47, wherein administration of the medication results in a pharmacokinetic profile characterized by a Cₘₐₓ of 850 pg/ml or less, and an AUC_{0-inf} that does not exceed 75,000 pg-min/ml.

52. A pharmaceutical composition for treating a respiratory condition, wherein a dose of the pharmaceutical composition comprises polysorbate 80, hydrogenated soy lecithin, EDTA, sodium chloride, sodium citrate dihydrate, citric acid and 0.25 mg or less of micronized budesonide, and results in an AUC_{0-inf} increases by more than 1.5 times the AUC_{0-inf} of the initial dose when the dose of the pharmaceutical composition is administered at least twice a day for at least 7 days, characterized in that the pharmaceutical composition is administered by nebulization and the nebulization time is 5.0 minutes or less.

53. The pharmaceutical composition of claim 52, wherein a Tₘₐₓ is the same time as the nebulization time of the pharmaceutical composition.

54. The pharmaceutical composition of claim 52, wherein the dose comprises 0.135 mg or less of micronized budesonide.

55. The pharmaceutical composition of claim 52, wherein the pulmonary condition is asthma.

56. The pharmaceutical composition of claim 52, wherein administration of the dose results in a pharmacokinetic profile characterized by a Cₘₐₓ of 850 pg/ml or less, and an AUC_{0-inf} that does not exceed 75,000 pg-min/ml.
